(19) United States
(12) Patent Application Publication
(10) Pub. No.: US 2004/0248788 A1
(43) Pub. Date: Dec. 9, 2004

(54) Hypertenson treatment

(76) Inventors: Mark Hedley Vickers, Auckland (NZ); Bernard Hermann Heinrich Breier, Auckland (NZ); Peter David Gluckman, Auckland (NZ)

Correspondence Address: FLIESLER MEYER, LLP, FOUR EMBARCADERO CENTER, SUITE 400, SAN FRANCISCO, CA, 94111, US

(21) Appl. No.: 10/482,854

(22) PCT Filed: Jul. 4, 2002
(86) PCT No.: PCT/NZ02/00118

(30) Foreign Application Priority Data
Jul. 6, 2001 (NZ) 512832

(51) Int. Cl. A61K 38/28

(52) U.S. Cl. 514/12

(57) ABSTRACT

A method of delaying or preventing the onset of hypertension in mammals, including predisposed mammals, is disclosed. The method includes the administering of an effective amount of or increasing the effective concentration of growth hormone, an analog thereof or a functionally equivalent ligand.

### TECHNICAL FIELD

This invention relates to a treatment that prevents or delays the onset of hypertension or high blood pressure in mammals, in particular those predisposed to such conditions.

### BACKGROUND ART

95% of all hypertension develops from unknown cause. This form of hypertension is often referred to as essential or primary hypertension to distinguish it from secondary hypertension that arises from known cause (i.e., due to specific illness or medical treatment) (Kaplan 1998).

Hypertension, or high blood pressure, affects about 50 million Americans and is a major risk factor for coronary heart disease, stroke, and heart failure (Centers for Disease Control and Prevention and National Institutes of Health “Heart Disease and Stroke” Healthy People 2010-Conference Edition, November 1999). Of those with high blood pressure, 36% do not realize they have it, thus, hypertension has been referred to as a “silent killer”. The prevalence of high blood pressure increases with age. However, in older age groups the disease is usually relatively mild compared to that in young adults where it is often more severe. Treatment of high blood pressure can prolong life.

Interestingly, clinical and epidemiological studies have found a strong correlation between intrauterine growth retardation (IUGR) and the onset of adult disease such as hypertension, but also obesity and type 2 diabetes (Barker 1994, 1996; Woodall et al 1996). So established is this connection that one estimate suggests that 45% of deaths from coronary heart disease would be avoided if the ‘fetal origin to adult disease’ component were removed (Reaven & Laws, 1999). The causes of IUGR are varied (poor maternal nutrition, maternal disease, and placental function), but have in common the interference of nutrient delivery to the fetus (Gluckman & Harding, 1997). “Programming” is the mechanism that explains the phenomenon of IUGR and fetal origin of adult disease. In short, experiences in the womb “program” fetal development As an example, poor nutrition in utero “programs” the fetus for an expectation of poor nutrition after birth, and vital organs and overall body plan develop accordingly. Abnormalities in organ development have been noted following poor nutrition in utero (Hoet et al 2000) and one can imagine that many other adverse scenarios—not just poor nutrition—could potentially lead to developmental programming that manifests as disease in later life (Barker 1994; Godfrey 1998).

In experimental animal models, it is reported that malnutrition in utero leads to hypertension in adult life (Persson & Jansson, 1992; Langley-Evans et al 1994). We have shown in a rodent model of intrauterine programming that maternal administration of growth hormone to pregnant rats on a reduced diet (30% of ad-libitum) could not prevent in their offspring the subsequent growth retardation or rise in systolic blood pressure, suggesting a mechanism of maternal nutrient partitioning at the expense of the fetus (Woodall et al 1999). However, in a similar experimental paradigm, treating the adult programmed offspring exhibiting high blood pressure with growth hormone, resulted in a significant decrease in systolic blood pressure (Vickers et al WO 00/30588).

The present invention is unexpected given our previous observation that adult offspring from ad-libitum control fed mothers showed no significant change in systolic blood pressure following treatment, and that reduction in systolic blood pressure was most marked in programmed animals already exhibiting symptoms of hypertension (Vickers et al WO 00/30588). Ad-libitum and programmed offspring treated with vehicle only showed no significant change in the systolic blood pressure exhibited (Vickers et al WO 00/30588). In addition, previous reports have associated an increase in systolic blood pressure with long-term exposure to endogenous growth hormone in acromegalics (Sacca et al 1994) and low systolic blood pressure after a long-term lack of exposure to endogenous growth hormone in growth hormone deficient adults (Sacca et al 1994). In other studies, growth hormone has been reported to decrease diastolic blood pressure (Johannsson et al 1997), while having no effect on systolic blood pressure.

More recently, growth hormone has been shown to be safe for long-term treatment in pre-pubescent children born short for gestational age (Sas et al 2000). In this study, children were administered recombinant human growth hormone on a daily basis for a period of up to 6 years. The authors reported favorable effects on body composition, systolic blood pressure, and lipid metabolism. A critical difference between our work and that of Sas et al (2000) is that in our experimental animal model we obtained results of long-term effects of growth hormone on blood pressure after treatment cessation Sas et al also make no comment on the use of growth hormone to prevent or delay symptoms of hypertension in individuals prone to that condition.

The rising costs of health care and the increased number of individuals suffering from hypertension provide ample reason for seeking to prevent or delay onset of disease rather than treating disease after it occurs.

### OBJECT OF INVENTION

It is an object of the present invention to provide a method of preventing or delaying the onset of hypertension in mammals, including those predisposed to such conditions, or at least to offer the public a useful choice of treatment.

### DISCLOSURE OF INVENTION

Accordingly, the present invention provides a method of preventing or delaying the onset of hypertension in a mammal, comprising the step of administering to the mammal an effective amount of, or increasing the effective concentration of, growth hormone, an analog thereof, or a functionally equivalent ligand, prior to onset of hypertension or symptoms thereof.

Preferably the mammal is a pre-pubescent or adult mammal prior to the onset of hypertension or symptoms thereof.

Preferably the mammal is human.

Preferably the mammal is predisposed to essential or primary hypertension.

Preferably the mammal is predisposed to essential or primary hypertension as a consequence of intrauterine fetal programming or intrauterine growth retardation.

Preferably the effective concentration of growth hormone, an analog thereof or a functionally equivalent ligand in the mammal is increased through administration of an agent which either stimulates production of growth hormone or which lessens or prevents inhibition of growth hormone activity.

In a further aspect, the present invention provides the use of growth hormone, an analog thereof, a functionally equivalent ligand or an agent which either stimulates production of growth hormone or which lessens or prevents inhibition of growth hormone activity in the preparation of a medicament for preventing or delaying the onset of hypertension in a mammal.

Preferably the medicament is administered or to be administered to a mammal predisposed to essential or primary hypertension.

Preferably the medicament is administered or to be administered to a mammal predisposed to essential or primary hypertension as a consequence of intrauterine fetal programming or intrauterine growth retardation.

Preferably the medicament is administered or to be administered to pre-pubescent or adult mammals prior to the onset of hypertension or the symptoms thereof.

In a further aspect the effective amount of growth hormone, an analog thereof, a functionally equivalent ligand or an agent which either stimulates production of growth hormone or which lessens or prevents inhibition of growth hormone activity is administered in a pharmaceutically acceptable combination with one or more suitable carriers or excipients.

In a further aspect the effective amount of growth hormone, an analog thereof, a functionally equivalent ligand or an agent which either stimulates production of growth hormone or which lessens or prevents inhibition of growth hormone activity is administered in combination with anti-hypertensive agents.

Preferably the anti-hypertensive agents are selected from the group including but not limited to ACE inhibitors or angiotensin II antagonists.

Preferably the ACE inhibitor is selected from a group that includes but is not limited to captopril, cilazapril, enalapril, fosinopril, imidapril, lisinopril, moexipril, perindopril, quinapril, ramipril, trandolapril.

Preferably the angiotensin II antagonist is selected from a group that includes but is not limited to candesartan, irbesartan, losartan, telmisartan and valsartan.

Preferably administration is by way of injection, implant, or administration of a replicable vehicle encoding for said growth hormone, an analog thereof, a functionally equivalent ligand or an agent which either stimulates production of growth hormone or which lessens or prevents inhibition of growth hormone activity.

Preferably the administration is through intravenous, oral, rectal, transdermal and/or nasal route.

It will be usual for the dosage range of administration of growth hormone, an analog thereof, a functionally equivalent ligand to be from about 0.1 microgram/kilogram/day to about 1 milligram/kilogram/day.

Preferably the dosage range of administration of growth hormone, an analog thereof, a functionally equivalent ligand is from about 2 to about 200 microgram/kilogram/day.

It will be usual for the increase in effective concentration of growth hormone, an analog thereof, a functionally equivalent ligand to be from about 0.1 microgram/kilogram/day to about 1 milligram/kilogram/day.

Preferably the increase in effective concentration of growth hormone, an analog thereof, a functionally equivalent ligand is from about 2 and to about 200 microgram/kilogram/day.

Although the invention is broadly defined above, it also includes embodiments of which the following description provides examples.

### BRIEF DESCRIPTION OF DRAWINGS

The invention will be better understood by way of example with reference to the accompanying drawings, in which:

FIG. 1: is a schematic depicting the timeline of the experimental paradigm. Treatment of young female rats with recombinant bovine growth hormone was initiated at postnatal day 25 administered twice daily until postnatal day 55; when growth hormone treatment was terminated. Rats were monitored for an additional 45 days after treatment (up to postnatal day 100). Systolic blood pressure recordings were taken at postnatal day 55, postnatal day 70, and postnatal day 100.

FIG. 2: is a graphical presentation of the change in blood pressure following growth hormone treatment. The nomenclature is as follows: ADCBS, female offspring from dams fed ad-libitum and treated with carbonate buffered saline; ADGH, female offspring from dams fed ad-libitum and treated with growth hormone; UNCBS, female offspring from dams fed 30% ad-libitum and treated with carbonate buffered saline; UNGH, female offspring from dams fed 30% ad-libitum and treated with growth hormone.

### DESCRIPTION OF THE INVENTION

As used herein, the term “intrauterine programming or growth retardation” means disordered fetal growth with causes including but not limited to maternal undernutrition, placental insufficiency, endocrine abnormalities and substance abuse.

As used herein, “analog” means a protein which is a variant of growth hormone. Preferably the growth hormone variants may be produced by insertion, deletion or substitution of one or more amino acids but which retains at least substantial functional equivalency. As used herein, “analog” includes synthetic analogs of growth hormone.

The term “functionally equivalent ligand” means an agent that binds to and activates the receptors which growth hormone binds to and activates to give the anti-hypertensive effect

The focus of the invention is on a form of treatment that prevents or delays the onset of hypertension. The surprising finding, which underlies the present invention, is that in a rodent model of intrauterine programming, administration of growth hormone of fixed duration to a pre-pubescent rat, prior to onset of symptoms, prevents or delays hypertension. More importantly, the prevention or delay of hypertension occurs regardless of whether there has been intrauterine programming or not. The benefits of administering growth hormone continue well after treatment has terminated and act to prevent and/or delay the onset of hypertension.

Such a finding is applicable towards a preventative course of treatment for individuals predisposed to essential or primary hypertension (i.e., due to family history); towards those individuals who may acquire hypertension due to adverse gestational conditions while still in the womb, known as “fetal or intrauterine programming”; or towards hypertension in general

The invention, broadly speaking, therefore provides a method of preventing or delaying the onset of hypertension in a mammal. That mammal may be predisposed to essential or primary hypertension or be one who has experienced intrauterine programming or growth retardation. It is however envisaged that the invention will have application in preventing or delaying mammalian hypertension caused by other etiologies, risk factors and environmental effects.

It is also envisaged that the principal application of the method of the invention will be to pre-pubescent or adult humans prior to onset of hypertension, although treatment of non-human mammals is in no way excluded.

In a preferred aspect, the method of the present invention involves administering to a mammal an effective amount of growth hormone, an analog thereof or a functionally equivalent ligand prior to onset of the symptoms of hypertension, to prevent or delay onset of those symptoms. In a preferred embodiment, growth hormone itself is administered to the mammal.

The growth hormone can be any mammalian growth hormone that will be known to a person skilled in this art, with examples being human growth hormone, bovine growth hormone, rat growth hormone and porcine growth hormone. It is, however, preferred that the growth hormone employed be human growth hormone where the mammal is a human. The growth hormone which is used in this invention can be obtained from any commercial source.

In addition to growth hormone itself, the use of analogs of growth hormone or functionally equivalent ligands of growth hormone is contemplated. By way of explanation, a protein is a functional equivalent of another protein for a specific function if the equivalent protein is immunologically cross-reactive with, and has at least substantially the same function as, the original protein. The equivalent can be, for example, a fragment of the protein, a fusion of the protein with another protein or carrier, or a fusion of a fragment with additional amino acids. For example, it is possible to substitute amino acids in a sequence with equivalent amino acids using conventional techniques. Groups of amino acids normally held to be equivalent are:

(a) Ala, Ser, Thr, Pro, Gly;

(b) Asn, Asp, Glu, Gln;

(c) His, Arg, Lys;

(d) Met, Leu, Ile, Val; and

(e) Phe, Tyr, Trp

It will also be appreciated that the present invention also extends to the administration of an agent which either stimulates production of growth hormone, or which lessens or prevents inhibition of growth hormone activity, i.e. to the administration of growth hormone agonists or secretagogues (substances which effect a direct increase in production of growth hormone).

Examples of agents that stimulate growth hormone and production or lessen or prevent its inhibition include, but are not limited to, growth hormone releasing peptides (GHRP) such as GHRP-1, GHRP-2, GHRP-6, hexarelin, G-7039, G-7502, L692,429, L-692,585, L-163,191 or growth hormone releasing hormone (GHRH) or inhibitors of growth hormone antagonists (substances which bind growth hormone or otherwise prevent or reduce the action of growth hormone within the body). These Latter compounds exert an indirect effect on effective growth hormone concentrations through the removal of an inhibitory mechanism, and include substances such as somatostatin release inhibitory factor (SRIF).

The active agent can be administered using any suitable route. Where growth hormone is the active compound to be administered, it will generally be administered as an injectable formulation, in combination with one or more suitable carriers or excipients. Those persons skilled in the art will appreciate how suitable formulations can be prepared.

The active agent can also be administered in combination. For example, a combination of growth hormone and other conventional anti-hypertensive agent(s), for example ACE (angiotensin-converting enzyme) inhibitors such as quinapril, or angiotensin II antagonists, such as losartan, is also contemplated. The ACE inhibitor may be selected from a group that includes but is not limited to captopril, cilazapril, enalapril, fosinopril, imidapril, lisinopril, moexipril, perindopril, quinapril, ramipril trandolapril. The angiotensin II antagonist may be selected from a group that includes but is not limited to candesartan, irbesartan, losartan, telmisartan and valsartan

Another possibility is administration of a replicable vehicle encoding the growth hormone/analog/ligand to the patient Such a vehicle (which may be a modified cell line or virus that expresses growth hormone/analog/ligand within the patient) could have application in increasing the concentration of the active compound within the patient for a prolonged period. Such a vehicle could well form part of an implant

Dosage levels will be formulation dependent. However, by way of example, the recommended dosage rate of growth hormone formulated for injection would be in the range of about 0.1 microgram/kilogram/day to about 1 milligram/kilogram/day. A preferred dosage rate would be from approximately about 2 to about 200 microgram/kilogram/day.

The method of the present invention will be effective in preventing or delaying the onset of hypertension, particularly in individuals predisposed towards this condition. The possibility of effective hormonal therapy for the hypertensive population is of immense public health significance.

The invention will now be further described with reference to the following non-limiting example.

All animal work described in the examples has been approved by the Animal Ethics Committee of the University of Auckland, New Zealand.

### EXAMPLE 1

Experimental

Virgin Wistar rats (age 80±5 days, n=15 per group) were time-mated using a rat oestrous cycle monitor (Fine Science tools INC., North Vancouver, BC, Canada) to assess the stage of oestrous of the animals prior to introducing the male. Day 1 of pregnancy was determined by the presence of spermatozoa after a vaginal smear. After confirmation of mating, rats were housed individually in standard rat cages containing wood shavings as bedding and with free access to water. All rats were kept in the same room with a constant temperature maintained at 25° C. and a 12-h light 12-h darkness cycle. Dams were randomly assigned to receive food either ad-libitum (n=30, 15 study animals and 15 dams for cross-fostering) or to receive 30% of ad-libitum (determined by measuring food intake on the previous day of an ad-libitum fed dam). The diet composition was protein 18%, fat 4%, fibre 3%, ash 7%, and carbohydrate 58% (Diet 86, Skellerup Stock Foods, Auckland, New Zealand). Food intake and body weights were recorded daily.

Following birth, offspring from restricted fed dams were cross-fostered onto ad-libitum fed mothers. Cross-fostering is necessary due to lactational insufficiency in restricted fed dams. Litter size was adjusted to 8 pups per litter to ensure standardized nutrition Pre-weaning weights of all pups were recorded daily. At weaning, (age 21 days) pups were sexed and housed in cages (females 2 per cage) and fed ad-libitum for the remainder of the study.

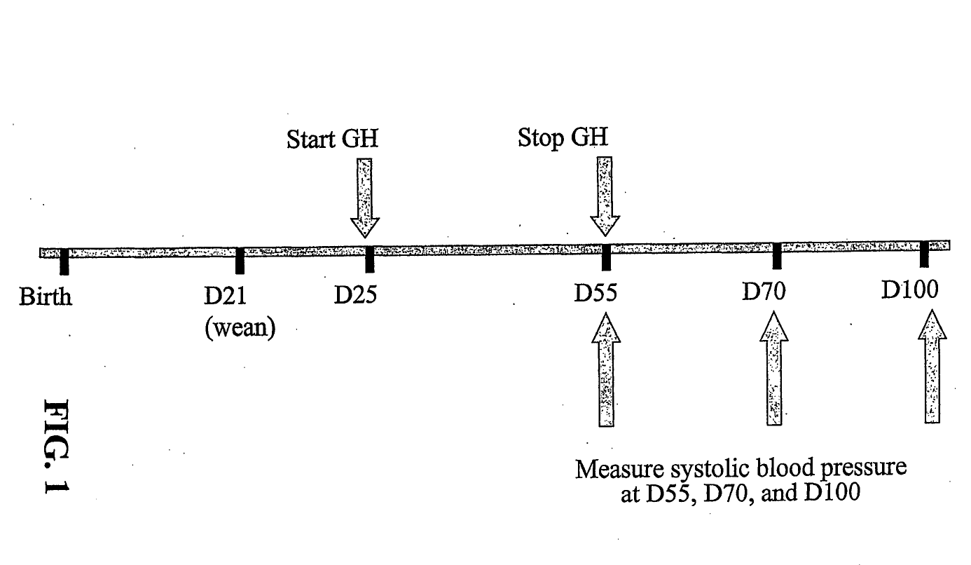

Treatment with recombinant bovine growth hormone (rbGH) commenced on postnatal day 25 and was administered at a dose of 10 microgram/gram bodyweight/day by twice daily subcutaneous injections (8 am and 5 pm) over a period of 30 days (FIG. 1). Growth hormone treatment was terminated on postnatal day 55 and the animals were monitored until postnatal day 100. Control animals were treated with carbonate buffered saline (CBS, pH 9.4) in place of growth hormone using an identical protocol.

Methods

Systolic blood pressures were recorded by tail cuff plethysmography according to the manufacturer's instructions (Blood pressure analyzer IITC, Life Science, Woodland Hills, Calif. USA). Systolic blood pressures was measured on postnatal days 55, 70, and 100 respectively. Rats were restrained in a clear plastic tube in a warmed room (25-28° C.). After the rats had acclimatized (10-15 min) the cuff was placed on the tail and inflated to 240 mmHg. Pulses were recorded during deflation at a rate of 3 mmHg/sec and reappearance of a pulse was used to determine systolic blood pressure. A minimum of 3 clear systolic blood pressure recordings were taken per animal. Coefficient of variation for repeated measurements was <5%.

Results

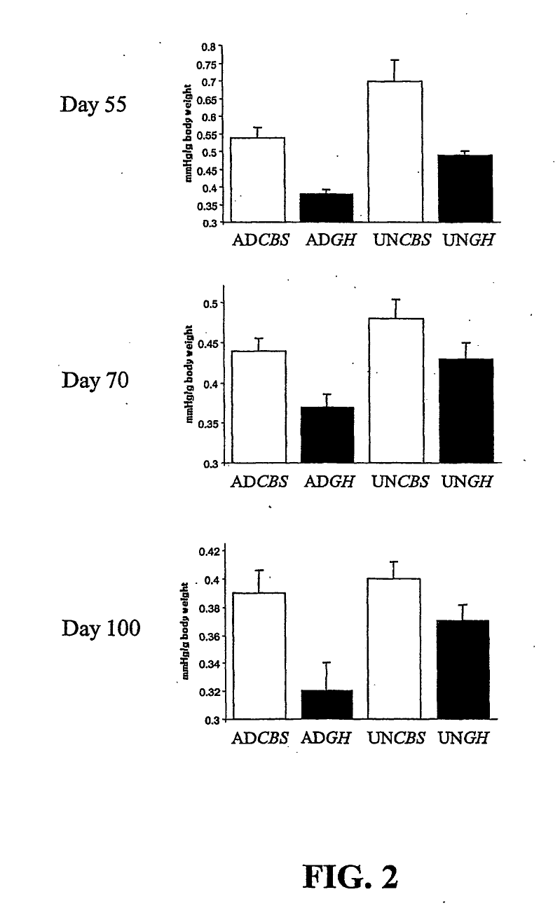

Systolic blood pressure was significantly decreased in all animals treated with rbGH for 30 days (FIG. 2). In general, we observed that offspring from undernourished dams (UNCBS) had higher blood pressure than offspring from ad-libitum fed dams (ADCBS); however, the UN group had not yet become critically hypertensive, as observed in adult programmed rats from previous studies (Vickers et al 2000). The reference standard for hypertension in the adult rodent is a systolic blood pressure (SBP) of greater than or equal to (≧) 150 mmHg (Capasso et al 1987). ADCBS rats typically had a SBP of 115 mmHg, and UNCBS rats a SBP of 125 mmHg prior to treatment We consider the AD group to have been normotensive prior to administration of saline or growth hormone.

### CONCLUSION

The above results clearly demonstrate the utilization of growth hormone as a mode of treatment to prevent or delay the onset of hypertension.

The mechanism of growth hormone action as observed here is unknown at this time. We also cannot rule out the possibility that growth hormone in younger animals acts to reduce blood pressure via an alternate mechanism(s) from what we observed previously in hypertensive adult rats.

Although the invention has been described with reference to particular embodiments, it will be appreciated by those persons skilled in the art that variations and modifications may be made without departing from the spirit and scope of the invention.

### REFERENCES

Barker D J (1994) Outcome of low birthweight. Horm Res 42: 223-230.

Barker D J (1996) Growth in utero and coronary heart disease. Nutr Rev 52: S1-S7.

Barker D (1998) Mothers, Babies and Health in Later Life. Churchill Livingstone, Edinburgh, 2nd edition.

Capasso J A, Strobeck J E, Sonnenblick E H (1981) Myocardial mechanical alterations during gradual onset long-term hypertension in rats. Am J Physiol 241: H435-41.

Glucklnan P D, Harding J E (1997) The physiology and pathophysiology of intrauterine growth retardation. Horm Res 48 (suppl): 11-16.

Godfrey K M (1998) Maternal regulation of fetal development and health in adult life. Eur J Obstet Gynecol Reprod Biol 78: 41-150.

Hoet J J, Ozanne S, Reusens B (2000) Influences of pre- and postnatal nutritional exposures on vascular/endocrine systems in animals. Environ Health Perspect 108 Suppl 3: 563-568.

Johannsson G et al (1997) Growth hormone treatment of abdominally obese men reduces abdominal fat mass, improves glucose and lipoprotein metabolism and reduces diastolic blood pressure. J Clin Endocrinol Metab 82: 727-734.

Kaplan, N M (1998) Primary hypertension: pathogenesis. In: Clinical Hypertension. Williams & Wilkins, Maryland, 7th edition, pp 41-99.

Langley-Evans S C, Welham S J M, Jackson A A (1999) Fetal exposure to a maternal low protein diet impairs nephrogenesis and promotes hypertension in the rat Life Sci 64: 965-974.

Laor A, Stevenson D K, Shemer J, Gale R, Seidman D S (1997) Size at birth, maternal nutritional status in pregnancy, and blood pressure at age 17: population based analysis. BMJ 315: 449-453.

Persson. E, Jansson T (1992) Low birth weight is associated with elevated adult blood pressure in the chronically catheterized guinea pig. Acta Physiol Scand. 145: 195-196.

Reaven G, Laws A (1999) In: Insulin resistance: the metabolic Syndrome X, Humana Press, 1999.

Sacca L et al (1994) Growth hormone and the heart. Endocrin Rev 15: 555-573.

Sas T, Mulder P, Hokken-Koelega A (2000) Body composition, blood pressure, and lipid metabolism before and during long-term growth hormone (GH) treatment in children with short stature born small for gestational age either with or without OH deficiency. J Clin Endocrinol Metab 85: 3786-3792.

Schoen F J (1994) Blood vessels. In: Robbins Pathologic Basis of Disease. Cotran R S, Kumar V, Schoen F (eds). Philadelphia: W. B. Saunders Company, pp 467-516.

Vickers M H, Breier B H, Cutfield W S, Hofman P L, Gluckman P D (2000) Fetal origins of hyperphagia, obesity, and hypertension and postnatal amplification by hypercaloric nutrition. Am J Physiol Endocrinol Metab 279: E83-E87.

Woodall S M et al (1996a) A model of intrauterine growth retardation caused by chronic maternal undernutrition in the rat: effects on the somatotrophic axis. J Endocrinol 150: 231-242.

Woodall S M, Johnston B K Breier B H, Gluckman P D (1996b) Chronic maternal undernutrition in the rat leads to delayed postnatal growth and elevated blood pressure of offspring. Pediatr Res 40: 438-443.

Woodall S M, Bassett N S, Gluclanan P D, Breier B H (1998) Consequences of maternal undernutrition for fetal and postnatal hepatic insulin-like growth factor-1, growth hormone receptor and growth hormone binding protein gene regulation in the rat. J Mol Endocrinol 20: 313-326.

Woodall S M, Breier B H, Johnston B M, Bassett N S, Barnard R, Gluckman P D (1999) Administration of growth hormone or IGF-1 to pregnant rats on a reduced diet throughout pregnancy does not prevent fetal intrauterine growth retardation and elevated blood pressure in adult offspring. J Endocrinol 163: 69-77.

1. A method of preventing or delaying the onset of hypertension in a mammal including the step of administering to the mammal an effective amount of or increasing the effective concentration of growth hormone prior to onset of hypertension, or a symptom thereof.

2. The method as claimed in claim 1 wherein said mammal is a pre-pubescent or adult mammal.

3. The method as claimed in claim 1 wherein said mammal is human.

4. The method of claim 3 wherein said mammal is predisposed to essential or primary hypertension.

5. The method as claimed in claim 4 wherein said predisposition results from intrauterine fetal programming or intrauterine growth retardation.

6. The method of claim 1 wherein said effective concentration of growth hormone, is increased through administration of an agent which either stimulates production of growth hormone or which lessens or prevents inhibition of growth hormone activity.

7. The use of growth hormone or an agent which either stimulates production of growth hormone or which lessens or prevents inhibition of growth hormone activity in the preparation of a medicament for preventing or delaying the onset of hypertension in a mammal.

8. The use as claimed in claim 7 wherein said mammal is predisposed to essential or primary hypertension.

9. The use as claimed in claim 8 wherein said predisposition is a consequence of intrauterine fetal programming or intrauterine growth retardation.

10. The use of claim 7 wherein the medicament is administered to pre-pubescent or adult mammals prior to the onset of hypertension or a symptom thereof.

11. The method of claim 1 wherein said effective amount of growth hormone or an agent which either stimulates production of growth hormone or which lessens or prevents inhibition of growth hormone activity is administered in a pharmaceutically acceptable combination with one or more suitable carriers or excipients.

12. The method as claimed in claim 11 wherein said effective amount of growth hormone is administered in combination with an anti-hypertensive agent.

13. The method as claimed in claim 12 wherein said anti-hypertensive agent is selected from the group consisting of angiotensin converting enzyme inhibitors or angiotensin II antagonists.

14. The method of claim 11 wherein said administration is by way of injection, implant, or administration of a replicable vehicle encoding for said growth hormone or an agent which either stimulates production of growth hormone or which lessens or prevents inhibition of growth hormone activity.

15. The method of claim 11 wherein said administration is through intravenous, oral, rectal, transdermal and/or nasal route.

16. The method of claim 1 wherein the dosage range of administration of said growth hormone is from about 0.1 microgram/kilogram/day to about 1 milligram/kilogram/day.

17. The method as claimed in claim 16 wherein the dosage range of administration is from about 2 to about 200 microgram/kilogram/day.

18. The method of claim 1 wherein said increase in effective concentration of growth hormone is from about 0.1 microgram/kilogram/day to about 1 milligram/kilogram/day.

19. The method as claimed in claim 18 wherein said increase in effective concentration is from about 2 and to about 200 micrograms/kilogram/day.

20-21. (Canceled)

22. A method for delaying or preventing symptoms of hypertension in a post-natal mammal destined to develop hypertension, comprising administering to said mammal a pharmaceutically effective amount of growth hormone.

23. The method of claim 22, wherein said mammal is post natal and has been subjected to intrauterine undernutrution.

24. The method of claim 22, wherein said mammal has been subjected to maternal disease or poor placental function.

25. The method of claim 1, wherein increasing the effective concentration of growth hormone is carried out by administering an agent selected from the group consisting of growth hormone releasing peptide-1 (GHRP-1), GHRP-2, GHRP-6, hexarelin, G-7039, G-7502, L-692,429, L-692,585, L-163,191 and somatostatin release inhibitory factor.

26. The method of claim 13, wherein said angiotensin-converting enzyme inhibitor is selected from the group consisting of quinapril, captopril, cilazapril, enalapril, fosinopril, imidapril, lisinopril, moexsipril, perindopril, ramipril and trandolapril.

27. The method of claim 13, wherein said angiotensin II antagonist is selected from the group consisting of candesartan, irbesartan, losartan, telmisartin and valsartin.
